(19) United States
(12) Patent Application Publication
(10) Pub. No.: US 2002/0086004 A1
(43) Pub. Date: Jul. 4, 2002

(54) Use of a defective recombinant adenovirus comprising a nucleic acid encoding an angiogenic factor for treating pulmonary hypertension

(76) Inventors: Serge Adnot, Saint Maur (FR); Didier Branellec, Lyon (FR)

Correspondence Address: Finnegan, Henderson, Farabow,, Garrett & Dunner, L.L.P., 1300 I Street, N.W., Washington, DC, 20005-3315, US

(21) Appl. No.: 09/983,885

(22) Filed: Oct. 26, 2001

Related U.S. Application Data
(63) Continuation of application No. PCT/FR00/01060, filed on Apr. 21, 2000.
(60) Provisional application No. 60/139,734, filed on Jun. 18, 1999.

(30) Foreign Application Priority Data
Apr. 26, 1999 (FR) 99/05,272

(51) Int. Cl. A61K 48/00; A61K 39/12

(52) U.S. Cl. 424/93.21; 424/204.1

(57) ABSTRACT

The present invention relates to the use of a vector which comprises a nucleic acid encoding an angiogenic factor for preventing, ameliorating or treating pulmonary hypertension. It also relates to specific pharmaceutical compositions which enable these vectors to be administered locally and efficiently.

The present invention relates to the use of a vector which comprises a nucleic acid encoding an angiogenic factor for preventing, ameliorating or treating pulmonary hypertension. It also relates to specific pharmaceutical compositions which enable these vectors to be administered locally and efficiently.

Pulmonary hypertension is a commonly occurring disorder which is fatal in its serious forms and which currently lacks any treatment apart from transplantation.

The disorder is generally characterized by an increase in pulmonary arterial resistance, which hinders right ventricular ejection and compromises cardiac output. Several functional and structural anomalies of the pulmonary vascular wall are involved in the development of pulmonary hypertension, including: hyperplasia of the smooth muscle cells, together with medial and intimal hypertrophy, a build-up of the extracellular matrix, and vascular rarefaction with reduction in peripheral capillary density.

The invention provides, for the first time, an efficient method for treating pulmonary hypertension. This method is based on using vectors which comprise a nucleic acid encoding an angiogenic factor.

While studies have been carried out on the involvement of various angiogenic factors, such as the fibroblast growth factors (FGFs) and vascular endothelium growth factor (VEGF) in the development of pulmonary hypertension, it has not so far been possible to elucidate the role of these factors.

The first growth factor which was selected for endothelial cells, i.e. VEGF, was identified in 1989. The studies which have been carried out since then have demonstrated the importance of VEGF in normal and pathological angiogenic processes. This factor has a powerful angiogenic effect in the rabbit cornea and the chick chorioallantoic membrane, which are two classical in vivo models of angiogenesis. VEGF is also known as the vascular permeability factor. VEGF is a heparin-binding, homodimeric glycoprotein which is 34-36 kDa in size whose structure includes a signal peptide, thereby enabling it to be secreted. The gene encoding VEGF is composed of 8 exons. Four peptide forms are generated by alternative slicing. In man, they respectively comprise 121,165,189 and 206 amino acids.

In adults, VEGF is expressed in a large number of normal tissues, for example, the heart and lungs. This expression is not necessarily associated with significant angiogenesis. The different forms of VEGF recognize two receptors which possess tyrosine kinase activity and which belong to the fms family: the Flt-1 receptor and the Flk-1 receptor. The Flt-1 receptor, i.e. (VEGFR)-1, is a high affinity receptor (10 pM). The KDR or flk-1 or (VEGFR)-2 receptor is a low affinity receptor (750 pM) which is thought to be responsible for the mitogenic effects of the peptide. One of the most remarkable aspects of the regulation of VEGF expression is its sensitivity to hypoxic conditions. Relatively brief periods of hypoxia have been shown to stimulate the in vitro expression of VEGF by cells in culture, for example, cardiomyocytes and smooth muscle cells. However, it is not known what role this factor plays in the development or prevention of pulmonary hypertension.

The family of vascular endothelium growth factors also comprises other molecules which can be used in the present invention, such as PIGF (placenta growth factor), VEGF-B, VEGF-C, VEGF-D and VEGF-E. In one embodiment of the present invention, the invention comprises a nucleic acid encoding VEGF or VEGF-B

VEGF-B is produced in a large number of adult tissues, for example, the heart, the skeletal muscle and the pancreas. Two forms of peptide are generated by alternative slicing and respectively comprise 167 and 186 amino acids. While VEGF-B stimulates proliferation of the endothelial cells, it does not bind to the VEGFR-2 receptor.

The family of fibroblast growth factors (FGFs) comprises a large number of representatives, and at least 14 members have been identified to date (for a review, see Birnbaum et al. Médecine et Science 13, p 392-396, 1997). Although the FGF-1 and FGF-2 forms are expressed in the cells of the pulmonary epithelium and within the vascular cells in the lung, no information is available with regard to the expression of these factors in relation to pulmonary hypertension or with regard to ability of these factors to induce proliferation of the endothelial cells and the smooth muscle cells within the lung, or with regard to the expression of these factors within the bronchial or alveolar epithelial cells in relation to different environmental conditions, for example, hypoxic conditions.

Unexpectedly, the Applicant has now demonstrated that the transfer, into the lung, of a nucleic acid encoding an angiogenic factor makes it possible to reduce pulmonary arterial pressure, and prevent right ventricular hypertrophy which is associated with pulmonary hypertension, with an efficiency which has never previously been equalled.

In order to study the effects of angiogenic factors in preventing and treating hypoxic pulmonary hypertension, the Applicant used rats which were in a state of chronic hypoxia as a model. Nucleic acids encoding angiogenic factors were transferred using recombinant vectors of the adenovirus type which were administered by means of intratracheal instillation. The results which were obtained show that expressing angiogenic factors, such as, for example, VEGF-B or FGF-1, within the lung reduces pulmonary arterial pressure and prevents right ventricular hypertrophy and remodeling of the pulmonary vascular system. The invention thus provides, for the first time, a method for efficiently treating pulmonary hypertension.

Of the various angiogenic factors which can be used within the context of the present invention, those which may particularly be mentioned are: members of the fibroblast growth factor (FGF) family, more specifically FGF-1, FGF-2, FGF-4 and FGF-5, the vascular endothelium growth factors, more specifically VEGF, VEGF-B, VEGF-C, VEGF-D, VEGF-D and PIGF (placenta growth factor), and the factors of the angiopoietin type (angiopoietin 1 and angiopoietin 2).

The invention relates to the use of a vector which comprises a nucleic acid encoding an angiogenic factor for preparing a pharmaceutical composition which is intended for preventing, ameliorating or treating pulmonary hypertension.

In one embodiment of the present invention, the angiogenic factor is an endothelial cell growth factor which is selected from the FGF or VEGF or angiopoietin family, or a combination of at least two factors which are selected from at least one of these families. Examples of advantageous combinations of angiogenic factors which may, for example, be mentioned are the combination which combines at least FGF-1 and VEGF, the combination which combines at least FGF-1 and VEGF-B, the combination which combines at least FGF-1 and antiopoietin 1, the combination which combines at least VEGF and antiopoietin 1 and the combination which combines at least VEGF-B and angiopoietin 1.

According to one particular embodiment, the endothelial cell growth factor is selected from FGF-1, FGF-2, FGF-4 or FGF-5, or their variants.

According to another embodiment, the endothelial cell growth factor is selected from VEGF, VEGF-B, VEGF-C, VEGF-D or VEGF-E, or their variants. For example, the endothelial cell growth factor is selected from VEGF or VEGF-B.

Within the meaning of the present invention, a “variant” of a polypeptide or a protein is understood as being any analog, fragment, derivative or mutated form which is derived from a polypeptide or a protein and which retains at least one biological function of said polypeptide or said protein. Different variants of a polypeptide or a protein can exist in the natural state. These variants can be allelic variations which are characterized by differences in the nucleotide sequence of the structural genes which encode the protein, or can result from differential slicing or post-translational modifications. These variants can be obtained by substituting, deleting, adding or modifying one or more amino acid residues. These modifications can be effected using any techniques known to the skilled person.

These variants are, for example, molecules which have a higher affinity for their binding sites, sequences which permit improved expression in vivo, molecules which exhibit a greater resistance to proteases, or molecules which possess a greater therapeutic efficacy or fewer side-effects or, possibly, novel biological properties.

Variants of FGF-1 include, for example, natural variants of FGF-1, such as the forms which are described in U.S. Pat. No. 4,868,113 and which comprise 154 amino acids, 140 amino acids or 134 amino acids. Variants of VEGF include, for example, VEGF₁₂₁, VEGF₁₆₅, VEGF₁₈₉ and VEGF₂₀₆ forms. Variants of VEGF-B include, for example, VEGF₁₈₆ and VEGF₁₆₇ forms. In one embodiment of the present invention, the invention comprises a nucleic acid encoding a variant chosen from FGF-1(21-154), VEGF₁₆₅, VEGF₁₈₆, and VEGF₁₆₇.

Other variants which can be used in the context of the invention are, for example, molecules in which one or more residues have been substituted, derivatives which have been obtained by deleting regions which are not, or not greatly, involved in the interaction with the binding sites under consideration or which express an undesirable activity, and derivatives which contain additional residues, such as a secretion signal or a junction peptide, as compared with the native sequence.

In the case of FGF-1, the nucleotide sequence encoding the angiogenic factor advantageously also contains a secretion signal which directs the synthesized FGF-1 into the secretary routes of the infected cells such that the FGF-1 which is synthesized is released more efficiently into the extracellular compartments and can activate its receptors. The secretion signal employed can be a heterologous secretion signal or even an artificial secretion signal. An example which may be mentioned is the secretion signal of human β interferon, which gives rise to substantial secretion of FGF-1.

The angiogenic factor-encoding DNA sequence which is used in the context of the present invention can be a cDNA, a genomic DNA (gDNA) or a hybrid construct which consists, for example, of a cDNA into which one or more introns is/are inserted. The DNA sequences can also be synthetic or semisynthetic sequences. A cDNA or gDNA is particularly advantageously used. For example, using a gDNA can give rise to improved expression in human cells.

Advantageously, the sequence encoding the angiogenic factor is placed under the control of signals which enable it to be expressed in the cells of the pulmonary epithelium. The signals may be heterologous expression signals, that is to say, signals which are different from those which are naturally responsible for expressing the angiogenic factor. The signals can, for example, be sequences which are responsible for expressing other proteins or else synthetic sequences. For example, these sequences can be promoter sequences of eucaryotic or viral genes. For example, the promoter sequences can be promoter sequences which are derived from the genome of the cell which it is desired to infect. Similarly, the promoter sequences can be promoter sequences which are derived from the genome of a virus, including the adenovirus which is employed. The E1A, MLP, CMV, LTR-RSV, etc. promoters may, for example, be mentioned in this regard. Furthermore, these expression sequences can be modified by adding activation sequences, regulatory sequences or sequences which permit tissue-specific expression. Thus, in one embodiment of the present invention, the invention comprises expression signals which are active specifically, or in the main, in the cells of the pulmonary epithelium, such that the DNA sequence is only expressed and only produces its effect when the vector has actually infected these cells; the promoter of the cytokeratin 18 gene may, for example, be mentioned in this regard.

The nucleic acid encoding one or more angiogenic factors is introduced into a vector. Within the meaning of the present invention, “vector” is understood as being any means which enables a nucleic acid to be transferred into a host cell, for example, within the lung, such as within the pulmonary epithelium. The term vector comprises viral and nonviral vectors for transferring a nucleic acid into a cell in vivo or ex vivo. For example, a vector type for implementing the invention can be a plasmid, a cosmid or any DNA which is not encapsidated by a virus, a phage, an artificial chromosome, a recombinant virus, etc. The vector may be a plasmid or a recombinant virus.

Of the vectors of the plasmid type, all the cloning or expression plasmids which are known to the skilled person and which generally contain an origin of replication may be mentioned. Plasmids which carry improved origins of replication or selection markers, such as those described, for example, in applications WO96/26270 and WO97/10343, may also be mentioned.

Of the vectors of the recombinant virus type, recombinant adeno-associated viruses, adenoviruses, retroviruses, herpesviruses and lentiviruses, or the SV40 virus may be mentioned. The construction of this type of recombinant virus which are defective for replication has been widely described in the literature, as have the infection properties of these vectors (see, for example, S. Baeck and K. L. March (1998), Circul. Research Vol. 82, pp 295-305), T. Shenk, B. N. Fields, D. M. Knipe, P. M. Howley et al. (1996), Adenoviridae : the viruses and their replication (in virology). Pp 211-2148, EDS—Ravenspublishers/Philadelphia, P. Yeh and M. Perricaudet (1997), FASEB Vol. 11, pp 615-623.

In one embodiment of the present invention, a recombinant virus for implementing the invention is a defective recombinant adenovirus.

The adenoviruses are linear, double-stranded DNA viruses having a size of approximately 36 kb (kilobases). The adenoviruses exist in different serotypes, whose structure and properties vary somewhat, but which exhibit comparable genetic organization. Generally, the recombinant adenoviruses can be of human or animal origin. For example, adenoviruses of human origin may comprise those classed in group C, for example, the adenoviruses of the 2 (Ad2), 5 (Ad5), 7 (Ad7) or 12 (Ad12) type. Also for example, adenoviruses of animal origin amy comprise adenoviruses of canine origin, for example, all the strains of the CAV2 adenoviruses [Manhattan strain or A26/61 (ATCC VR-800), for example]. Other adenoviruses of animal origin are for example, mentioned in application WO94/26914, which is hereby incorporated by reference.

The adenovirus genome comprises, for example, an inverted repeat sequence (ITR) at each end, an encapsidation sequence (Psi), early genes and late genes. The main early genes are contained in the E1, E2, E3 and E4 regions. Of these, the genes contained in the E1 region are for example, required for viral propagation. The main late genes are contained in the L1 to L5 regions. The genome of adenovirus Ad5 has been fully sequenced and is accessible on databases (see, for example, Genbank M73260). Similarly, parts, if not the whole, of other adenoviral genomes (Ad2, Ad7, Ad12, etc.) have also been sequenced.

Various adenovirus-derived constructs, incorporating different therapeutic genes, have been prepared for their use as recombinant vectors. In each of these constructs, the adenovirus has been modified so as to render it incapable of replicating in the infected cell. Thus, the constructs described in the prior art are adenoviruses deleted from the E1 region, which is essential for viral replication, with the heterologous DNA sequences being inserted in place of this region (Levrero et al, Gene 101 (1991) 195; Gosh-Choudhury et al., Gene 50 (1986) 161). Furthermore, the creation of other deletions or modifications in the adenovirus genome has been proposed for improving the properties of the vector. Thus, a temperature-sensitive point mutation has been introduced in the mutant ts125, with this point mutation inactivating the 72 kDa DNA-binding protein (DBP) (Van der Vliet et al., J. Virol., 1975, 15(2) 348-354). The E4 region, which is essential for replication or viral propagation, has been deleted from other vectors. Thus, the E4 region is involved in regulating the expression of late genes, in the stability of the late nuclear RNAs, in extinguishing the expression of the proteins of the host cell and in the efficiency of the replication of the viral DNA. Adenoviral vectors from which the E1 and E4 regions have been deleted therefore exhibit a very reduced background noise of transcription and expression of the viral genes. Such vectors have been described, for example, in applications WO94/28152, WO95/02697 and WO96/22378. Furthermore, vectors which carry a modification within the IVa2 gene have also been described (WO96/10088).

In one embodiment of the present invention, the recombinant adenovirus is a human group C adenovirus. For example, the adenovirus can be chosen from an Ad2 or Ad5 adenovirus.

In another embodiment of the present invention, the recombinant adenovirus which is used within the context of the invention contains a deletion in the E1 region of its genome. For example, it may contain a deletion of the E1a and E1b regions. Deletions which affect nucleotides 454-3328, 386-3446 or 357-4020 (with reference to the Ad5 genome) may be mentioned by way of example.

According to another embodiment of the present invention, the recombinant adenovirus which is used in the context of the invention additionally contains a deletion in the E4 region of its genome. For example, the deletion in the E4 region affects all the open reading frames. The 33466-35535 or 33093-35535 deletions may be mentioned as specific examples. Other types of deletion in the E4 region are described in applications WO95/02697 and WO96/22378, which are hereby incorporated by reference.

The expression cassette which contains the nucleic acid encoding an angiogenic factor can be inserted at various sites in the recombinant genome. It can be inserted within the E1, E3 or E4 region, either while replacing the deleted sequences or in addition to the existing sequences. The expression cassette can also be inserted at any other site apart from the sequences which are required in cis for producing viruses (ITR sequences and encapsidation sequence).

Within the meaning of the present invention, a “cassette for expressing” a nucleic acid is understood as being a DNA fragment which can be inserted into a vector at specific restriction sites; besides the nucleotide sequence encoding an RNA or a polypeptide of interest, the DNA fragment comprises the sequences (enhancer(s), promoter(s), polyadenylation sequence, etc.) which are required for expressing the said sequence of interest. The DNA fragment and the restriction sites are devised for ensuring that said fragment is inserted into a reading frame which is appropriate for the transcription or the translation.

The recombinant adenoviruses are produced in an encapsidation cell line, that is a line of cells which are able to complement in trans one or more of the functions which are deficient in the recombinant adenoviral genome. An example of the encapsidation cell lines which are known to the skilled person which may be mentioned is cell line 293, into which a part of the adenovirus genome has been integrated. More specifically, cell line 293 is a line of human kidney embryonic cells which contains the left-hand end (approximately 11-12%) of the adenovirus serotype 5 (Ad5) genome, comprising the left-hand ITR, the encapsidation region, the E1 region, including E1a and E1b, the region encoding the pIX protein and a part of the region encoding the pIVa2 protein. This cell line is able to transcomplement recombinant adenoviruses which are defective for the E1 region, that is which lack all or part of the E1 region, and to produce high titers of viral stocks. This cell line is also able to produce, at the permitted temperature (32° C.), virus stocks which additionally contain the temperature-sensitive E2 mutation. Other cell lines which are able to complement the E1 region, and which are based, for example, on human lung carcinoma A549 cells (WO94/28152) or on human retinoblasts (Hum. Gen. Ther. (1996) 215) have been described. Furthermore, cell lines capable of transcomplementing several functions of the adenovirus have also been described. Cell lines which complement the E1 and E4 regions (Yeh et al., J. Virol. Vol. 70 (1996) pp 559-565; Cancer Gen. Ther. 2 (1995) 322; Krougliak et al., Hum. Gen. Ther. 6 (1995) 1575) and cell lines which complement the E1 and E2 regions (WO94/28152, WO95/02697 and WO95/27071) may for example, be mentioned.

The recombinant adenoviruses are normally produced by introducing viral DNA into the encapsidation cell line, with the cells then being lysed after approximately 2 or 3 days (since the kinetics of the adenoviral cycle are from 24 to 36 hours). In order to implement the method, the viral DNA which is introduced can be the complete recombinant viral genome, which can have been constructed in a bacterium (WO96/25506) or in a yeast (WO95/03400), and which has been transfected into the cells. The viral DNA can also be that of a recombinant virus which has been used for infecting the encapsidation cell line. The viral DNA can also be introduced in the form of fragments, each of which carries a part of the recombinant viral genome and a region of homology which makes it possible, following introduction into the encapsidation cell, to reconstitute the recombinant viral genome by means of homologous recombination between the different fragments.

After the cells have lysed, the recombinant viral particles are isolated by centrifugation in a cesium chloride gradient. An alternative method has been described in application WO98/00528, which is hereby incorporated by reference.

An example of a vector which is suitable for implementing the present invention and which may for example, be mentioned is: the recombinant adenovirus which comprises the gene encoding human FGF-1 or human VEGF-B, as described in the present invention, or the recombinant adenovirus which comprises the gene encoding the 165 isoform of human VEGF, as described in Mülhauser J et al. (VEGF 165 expressed by a replication-deficient recombinant adenovirus vector induces angiogenesis in vivo. Circ Res. 195;77:1077-1086) which is hereby incorporated by reference.

The invention also relates to a pharmaceutical composition which comprises a vector, as described above, and a physiologically acceptable excipient. The pharmaceutical compositions of the invention can be formulated with a view to administration by the oral, parenteral, intranasal, intraarterial, intravenous, etc. route.

In one embodiment of the present invention, the pharmaceutical composition comprises excipients which are pharmaceutically acceptable for a formulation which is intended to be administered by the intratracheal route, for example, by means of instillation, or by the intravenous route. The excipients may, for example, be sterile, isotonic saline (monosodium or disodium phosphate, sodium, potassium, calcium or magnesium chloride, etc. or mixtures of such salts) solutions, or dry, for example, lyophilized, compositions which, by means of the addition of sterilized water or physiological saline, as the case may be, enable solutions to be constituted, which are intended for intratracheal instillation.

The doses employed for the instillation or the injections may be adjusted in dependence on various parameters, for example, in dependence on the mode of administration employed, on the gene to be expressed or else on the sought-after duration of expression. In a general manner, the recombinant viruses according to the invention are formulated and administered in the form of doses containing between 10⁴ and 10¹⁴ pfu, preferably from 10⁶ to 10¹⁰ pfu. The term pfu (plaque forming unit) corresponds to the infectious capacity of a viral solution and is determined by infecting an appropriate cell culture and measuring the number of plaques of infected cells. The techniques for determining the pfu titer of a viral solution are well documented in the literature.

In addition, the compositions according to the invention can also comprise a chemical or biochemical transfer agent. The term “chemical or biochemical transfer agent” is understood as being any compound (i.e. other than a recombinant virus) which facilitates the penetration of a nucleic acid into a cell. The agents can also be cationic non-viral agents, such as cationic lipids, peptides, polymers (polyethylene imine, polylysine) or nanoparticles; or non-cationic non-viral agents, such as non-cationic liposormes, polymers or non-cationic nanoparticles.

According to one embodiment, the compositions according to the invention comprise a defective recombinant vector which comprises a gene which encodes an endothelial cell growth factor, and are formulated for an intratracheal administration. Advantageously, the compositions of the invention comprise from 10⁴ to 10¹⁴ pfu, such as from 10⁶ to 10¹⁰ pfu.

The invention also relates to a process for preparing a medicament which can be used for preventing, ameliorating or treating pulmonary hypertension, wherein a recombinant vector which comprises a nucleic acid encoding a growth factor is mixed with one or more compatible and pharmaceutically acceptable adjuvants.

The invention also relates to a method for treating a mammal, for example, an individual human, suffering from pulmonary hypertension, which method comprises administering an effective quantity of a recombinant vector which comprises a nucleic acid encoding an endothelial cell growth factor.

The present invention will be described in more detail with the aid of the examples which follow and which should be regarded as being illustrative and not limiting.

### LEGEND TO THE FIGURES

FIG. 1: obtaining the plasmid pXL3264, which is generated by double recombination from the plasmids pXL3208 and pXL3215, in accordance with the method described by Crouzet et al. (PNAS Vol. 94 p1414, 1997). Plasmid pXL3264 contains the genome of a type 5 adenovirus which has been deleted for the E1 and E3 regions, and contains the CMV-spFGF1-SV40 expression cassette.

FIG. 2 : diagram of the vector pXL3179. Plasmid pXL3179 is a vector which is derived from the plasmid pXL2774 (WO97/10343), into which the gene encoding a fusion between the signal peptide of human fibroblast interferon and the cDNA for FGF1 (fibroblast growth factor1)(sp-FGF1, Jouanneau et al., 1991 PNAS 88:2893-2897) has been introduced under the control of the promoter derived from the human cytomegalovirus early region (hCMV IE) and the polyadenylation signal of the SV40 virus late region (Genbank SV4CG).

FIG. 3 : diagram of the vectors pXL3636 and pXL3637. These vectors have structures which are comparable to that of plasmid pXL3208 and respectively contain sequences encoding VEGF-B₁₆₇ (pXL3636) and VEGF-B₁₈₆ (pXL3637) in place of the sp-FGF-1 (pXL3208, FIG. 1).

### MATERIAL AND METHODS

### General Molecular Biology Techniques

The methods which are conventionally employed in molecular biology, such as preparative extractions of plasmid DNA, the centrifugation of plasmid DNA in a cesium chloride gradient, electrophoresis on agarose or acrylamide gels, the purification of DNA fragments by electroelution, the extraction of proteins with phenol or phenol-chloroform, the precipitation of DNA in a saline medium with ethanol or isopropanol, transformation into Escherichia coli, etc., are well known to the skilled person and amply described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987.

For ligations, the DNA fragments can be separated, in accordance with their size, by electrophoresis in agarose or acrylamide gels, extracted with phenol or with a phenol/chloroform mixture, precipitated with ethanol and then incubated in the presence of phage T4 DNA ligase (Biolabs) in accordance with the supplier's recommendations.

The protruding 5′ ends can be filled in using the Kienow fragment of E. coli DNA polymerase I (Biolabs) in accordance with the supplier's specifications. The protruding 3′ ends are destroyed in the presence of phage T4 DNA polymerase (Biolabs), which is used in accordance with the manufacturer's recommendations. The protruding 5′ ends are destroyed by careful treatment with S1 nuclease.

In vitro site-directed mutagenesis using synthetic oligodeoxynucleotides can be carried out in accordance with the method developed by Taylor et al. [Nucleic Acids Res. 13 (1985) 8749-8764] using the kit distributed by Amersham.

DNA fragments can be amplified enzymatically by means of the PCR technique [polymerase-catalyzed chain reaction, Saiki R.K. et al., Science 230 (1985) 1350-1354: Mullis K. B. and Faloona F. A., Meth. Enzym. 155 (1987) 335-350] using a DNA thermal cycler (Perkin Elmer Cetus) in accordance with the manufacturer's specifications.

The nucleotide sequences can be verified by the method developed by Sanger et al [Proc. Natl. Acad. Sci. USA, 74 (1977) 5463-5467] using the kit distributed by Amersham.

### EXAMPLE 1

### Construction of Recombinant Adenoviruses which Express the FGF-1 Protein.

This example describes the construction of an adenoviral vector which carries the gene encoding the FGF-1 protein operationally linked to the CMV promoter.

The human cDNA encoding human FGF-1 encompasses 490 base pairs and encodes a 154 amino acid polypeptide which is also called ECGFβ, standing for beta-endothelial cell growth factor. Two natural polypeptides exist which were derived from the ECGFβ form by means of a post-translational maturation mechanism; these were acidic FGF (aa 15 to 154) and ECGFα (alpha-endothelial cell growth factor; aa 21 to 154) or FGF-1.

The FGF-1 form (sp-FGF-1) which is present in the adenovirus which is described below is in actual fact a protein formed by the fusion of FGF-1 (aa 21-154) and a human beta interferon signal peptide which is described by Jouanneau et al. (PNAS (1991) 88: 2893-2897).

The sp-FGF-1 was expressed under the control of the cytomegalovirus enhancer/promoter (CMV, nucleotides −522 to +72) (Boshart et al. 1985, Cell, 41:521-530). The SV40 virus polyadenylation site (nucleotides 2538 to 2759 in accordance with the SV40 genome, Genbank locus SV4CG) was inserted, in the late sense, into 3′ of the sp-FGF-1 cDNA. The overall unit formed by (i) the cytomegalovirus enhancer/promoter, (ii) the sp-FGF-1 cDNA and (iii) the SV40 virus polyadenylation site is termed the FGF-1 expression cassette below.

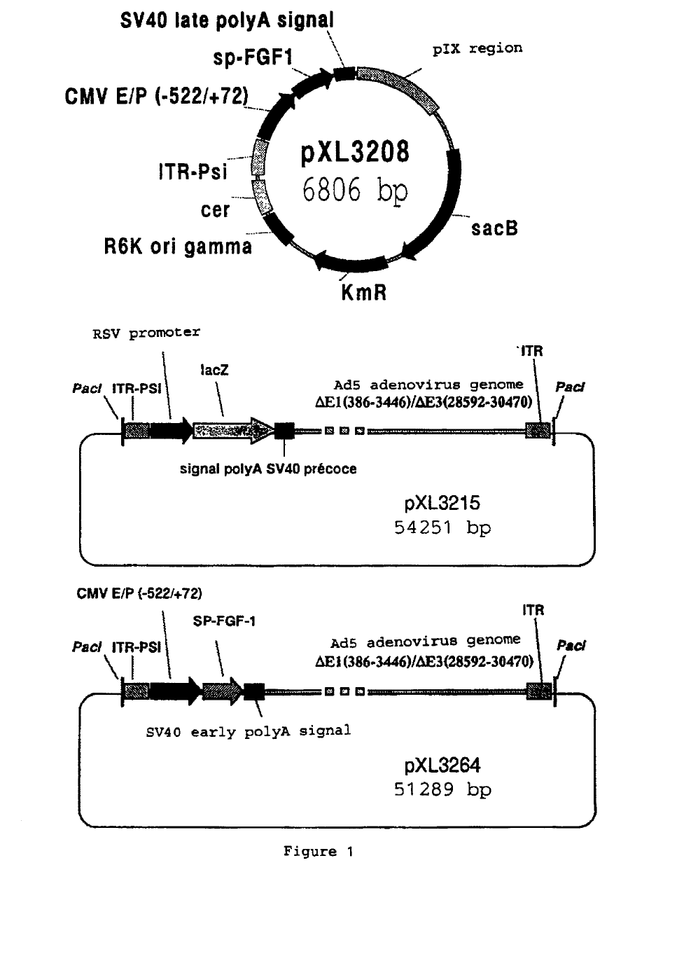

The adenovirus was constructed, in accordance with the method described by Crouzet et al. (PNAS Vol. 94 p1414, 1997), by means of homologous recombination between plasmid pXL3208 and plasmid pXL3215. Plasmid pXL3215 contained the genome of an adenovirus which contained an RSV-LacZ cassette inserted in its E1 region. The principle of the construction is depicted in FIG. 1. Plasmid pXL3264, which was generated by this double recombination, contained the genome of a type 5 adenovirus which was deleted for the E1 and E3 regions and which contained the CMV-spFGF-1-SV40 expression cassette. This construct was verified by sequencing the FGF-1 expression cassette.

The adenovirus AV1.0 CMV-FGF1 was generated by transfecting the Pacl-digested pXL3264 DNA into cell line 293 (ATCC CRL-1573). The viral particles which were obtained were then amplified in this same cell line and stocks of virus were produced by means of a double CsCI gradient.

The viral particles were then used for studying the expression of the human spFGF1 gene in C2C12 cells, mouse myoblast cells (ATCC CRL-1772) or W162 cells (Weinberg D. H. and Ketner G. A. 1983, PNAS 80:5383-5386).

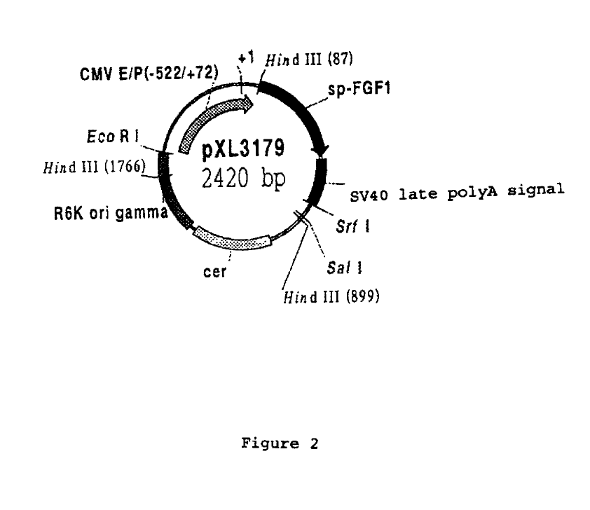

A Northern blot was carried out on the W162 cells after infecting at increasing MOIs of from 100 to 3000 viral particles (vp)/cell, or transfecting cells in the presence of lipofectamine (Gibco-BRL) with plasmid pXL3179, which contained the same FGF-1 expression cassette, as a control. Plasmid pXL3179 is depicted in FIG. 2.

A Western blot was carried out on the C2C12 cells after infecting at MOls of 30 to 3000 and harvesting the supernatants after 48 h. The FGF1 was visualized using a purified rabbit polyclonal anti-FGF1 antibody followed by a goat anti-rabbit antibody which was conjugated to peroxidase. The peroxidase activity was then visualized by chemiluminescence (ECL, Amersham) and detected on a Lumi-Imager (Roche diagnostics).

The culture supernatant from the C2C12 cells was used to verify that the expressed form of FGF was biologically active. Serial dilutions (1/200 and 1/50) of this supernatant were then added to cultures of NIH 3T3 cells. The trophic effect on these cultures was monitored by incorporating radiolabeled thymidine.

Taken overall, the results obtained confirmed that the adenovirus AV1.0 CMV-FGF1 expressed a biologically active form of FGF-1.

### EXAMPLE 2

### Construction of Recombinant Adenoviruses which Express the VEGF-B Protein

This examples describes the construction of an adenoviral vector which carries the gene encoding the VEGF-B protein operationally linked to the CMV promoter.

Two forms of human VEGF-B exist: i.e. VEGF-B₁₆₇ and VEFG-B₁₈₆, whose corresponding nucleotide sequences were accessible in Genbank under references HSU48801 (VEGF-B₁₆₇) and HSU43368 (VEFG-B₁₈₆).

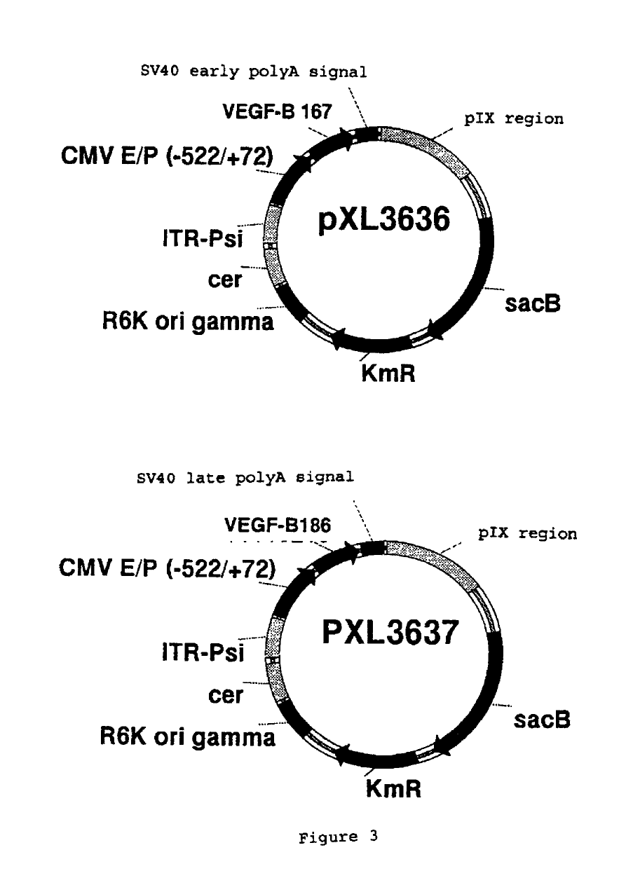

The adenoviruses AV1.0-CMV-VEGF-B₁₆₇ and AV1.0-CMV-VEFG-B₁₈₆ were constructed, in a manner similar to vector AV1.0-CMV-spFGF-1, from vectors pXL3636 and pXL3637 (FIG. 3). The expression of VEGF-B₁₆₇ or VEFG-B₁₈₆ was placed under the control of the cytomegalovirus enhancer/promoter (CMV, nucleotides −522 to +72) (Boshart et al. 1985, Cell, 41:521-530). The SV40 virus polyadenylation site (nucleotides 2538 to 2759 in accordance with the SV40 genome, Genbank locus SV4CG) was inserted, in the late sense, into 3′ of the VEGF-B₁₆₇ or VEFG-B₁₈₆ cDNA.

The adenovirus was constructed in accordance with the method described by Crouzet al. (PNAS Vol. 94 p 1414, 1997) by means of homologous recombination between plasmid pXL3636 (VEGF-B₁₆₇) and plasmid pXL3215, or between plasmid pXL3637 (VEFG-B₁₈₆) and plasmid pXL3215. Plasmid pXL3215 contained the genome of an adenovirus which contained an RSV-LacZ cassette inserted in its E1 region. The principle of the construction is depicted in FIG. 1.

The plasmid which was generated by this double recombination contained the genome of a type 5 adenovirus which was deleted for the E1 and E3 regions and contained either the CMV-VEGF-B₁₆₇-SV40 expression cassette or the CMV-VEGF-B₁₈₆-SV40 expression cassette.

The AV1.0-CMV-VEGF-B₁₆₇ and AV1.0-CMV-VEFG-B₁₈₆ adenoviruses were generated by transfecting the Pacl-digested plasmids, which were generated by the double recombination into the 293 cell line (ATCC CRL-1573). The viral particles which were obtained were then amplified in this same cell line, and viral stocks were produced using a double CsCI gradient.

### EXAMPLE 3

### Intrapulmonary Transfer of AV1.0-CMV-FGF1 in the Rat.

This example describes the transfer of the gene encoding human FGF-1 in the rat, using the above-described vector AV1.0 CMV-FGF1. A recombinant adenovirus which was identical but which did not contain the FGF-1 expression cassette (AV1.0 CMV.Null), was used in the control animals.

One-month-old male Wistar rats (200-250 g) were randomly divided into two groups. After having been anesthetized by the intraperitoneal injection of a mixture of ketamine (100 mg/kg) and xylasine (2 mg/kg), they were given either vector AV1.0 CMV-FGF1 or the control vector AV1.0 CMV.Null by instilling intratracheally with a dose of 10⁸ pfu (diluted in PBS to give a final volume of 150 μl). This dose was chosen after carrying out a dose-response study which comprised assaying the FGF-1 protein by ELISA in the bronchoalveolar lavage liquid and in the serum of the animals. 48 hours after the virus has been administered, the animals were exposed to an hypoxic gas mixture (10% Fio2) under standard pressure conditions (Flufrance cabinet, Cachan, France) for a period of 15 days.

### EXAMPLE 4

### Intrapulmonary Transfer of AV1.0-CMV-VEGF-B₁₆₇ and AV1.0-CMV-VEFG-B₁₈₆ in the Rat.

AV1.0-CMV-VEGF-B₁₆₇ and AV1.0-CMV-VEFG-B₁₈₆ were transferred into the lungs of the rat under conditions which were identical to those described in Example 3. A recombinant adenovirus which did not contain the VEGF-B expression cassette (AV1.0 CMV.Null) was used in the control animals.

### EXAMPLE 5

### Assessment of the Efficacy of the Intrapulmonary Transfer, in the Rat, of the Gene Encoding VEGF-B.

The assessment took place, after 15 days of exposure to hypoxia, in accordance with the criteria listed below.

a—Assessing the efficacy of transduction by means of assaying (ELISA) the FGF-1 protein in the bronchoalveolar lavage liquid.

b—Assessing the pulmonary hypertension by carrying out a hemodynamic study, by means of catheterizing the heart under anesthesia, with pulmonary arterial pressure, systemic arterial pressure and heart rate being measured.

c—Assessing right ventricular hypertrophy by calculating Fulton's index: weight of right ventricle weight of left ventricle+septum

d—Carrying out a histological and histomorphometric study of the lungs, with assessment of the degree of muscularization of the pulmonary arterioles at the alveolar and alveolar duct (diameter <200 μm) level.

### 2a—Efficiency of the Transduction

The human VEGF-B protein was assayed by ELISA in the serum and in the bronchoalveolar liquid (LBA) of the animals after 15 days of exposure to hypoxia. The VEGF-B factor was not found in the serum of any of the control animals whatever the conditions employed (i.e. with or without hypoxia). Similarly, the concentration of VEGF-B was zero in the LBA of the animals treated with the defective recombinant adenoviruses encoding VEGF-B (form 167 or form 186).

### 2b—Assessing the Pulmonary Hypertension

The pulmonary hypertension was assessed by carrying out a hemodynamic study, by catheterizing the heart under anesthesia, with pulmonary arterial pressure, systemic arterial pressure and heart rate being measured.

The body weights were identical in the two groups of rats 15 days after beginning exposure to hypoxia. The pulmonary arterial pressure in the rats treated with AV1.0-CMV-VEGF-B₁₆₇ or AV1.0-CMV-VEGF₁₈₆ was significantly less elevated than in the rats which were given the AdCMV.Null, while the systemic arterial pressure and the heart rate were not significantly different between the two groups (treated and control).

These results show that transferring a gene encoding VEGF-B very efficiently prevented the increase in arterial pressure which is linked to hypoxia.

### 2c—Assessing the Right Ventricular Hypertrophy

The right ventricular hypertrophy was assessed by determining the ratio of the weight of the right ventricle to that of the left ventricle+septum.

The results obtained show that the right ventricular hypertrophy was significantly greater in the rats given AV1.0 CMV.Null than in those treated with AV1.0 CMV-VEGF-B₁₆₇ or AV1.0 CMV-VEGF₁₈₆.

### 2d—Histological Study of the Lungs

The histological study of the lungs showed that the inflammatory lesions were very limited at the dose of 10⁸ pfu: absence of edema and hemorrhage, and absence of lesions in the bronchial or alveolar epithelia. Whatever the treatment administered, an interstitial granuloma, with macrophages dominating, was often observed.

The lungs were studied histomorphometrically in accordance with two criteria (i) studying the thickness of the arterial wall (arterioles having a diameter <200 μm), as standardized to the size of the artery, and (ii) studying the percentage of non-muscularized, partially muscularized or completely muscularized arteries at the alveolar and alveolar duct level.

The thickness of the arterial wall was determined, and the results obtained show that the thickness of the arterial wall, when standardized to the size of the artery, was significantly less in the rats given vector AV1.0 CMV.VEGF-B than in those treated with AV1.0 CMV.Null.

The percentage of non-muscularized, partially muscularized or completely muscularized arteries at the alveolar and alveolar duct level was determined, and the results obtained show that the percentage of non-muscularized arteries was significantly higher in the rats treated with the defective recombinant adenovirus vectors encoding VEGF-B, both at the alveolar level and at the alveolar duct level.

These results clearly demonstrate that over-expression of an endothelial cell growth factor, such as VEGF-B, in the lungs protected against the development of hypoxic pulmonary hypertension.

### EXAMPLE 6

### Assessing the Efficacy of the Intrapulmonary Transfer of the Human FGF-1 Gene in the Rat.

The assessment was carried out, after 15 days of exposure to hypoxia, in accordance with the criteria listed below.

a—Assessing the efficacy of transduction by assaying (ELISA) the FGF-1 protein in the bronchoalveolar lavage liquid.

b—Assessing the pulmonary hypertension by carrying out a hemodynamic study, by catheterizing the heart under anesthesia, with pulmonary arterial pressure, systemic arterial pressure and heart rate being measured.

c—Assessing right ventricular hypertrophy by calculating Fulton's index: weight of the right ventricle/weight of the left ventricle+septum

d—A histological and histomorphometric study of the lungs, with assessment of the degree of muscularization of the pulmonary arterioles at the alveolar and alveolar duct (diameter <200 μm) level.

The results obtained show that over-expression of an endothelial cell growth factor such as FGF-1 in the lungs protected against the development of hypoxic pulmonary hypertension.

What is claimed is:

1. A method of preventing, ameliorating or treating pulmonary hypertension in an individual comprising administering to said individual a vector which comprises a nucleic acid encoding at least one angiogenic factor, and expression of a therapeutically effective amount of said angiogenic factor in said individual.

2. The method according to claim 1, wherein said angiogenic factor is an endothelial cell growth factor chosen from the FGF family of growth factors and the VEGF family of growth factors.

3. The method according to claim 2, wherein said endothelial cell growth factor is chosen from FGF-1, FGF-2, FGF-4, FGF-5, and variants thereof.

4. The method according to claim 2, wherein said endothelial cell growth factor is chosen from VEGF, VEGF-B, VEGF-C, and variants thereof.

5. The method according to claim 1, wherein said vector is a plasmid, a cosmid, or a DNA which is not encapsidated by a virus.

6. The method according to claim 1, wherein said vector is a recombinant virus,

7. The method according to claim 6, wherein said vector is a recombinant virus derived from an adenovirus, a retrovirus, a herpesvirus, or an adeno-associated virus.

8. The method according to claim 6, wherein said recombinant virus is a defective recombinant adenovirus.

9. The method according to claim 6, wherein said recombinant virus is administered by the intratracheal route and said recombinant virus comprises from 10⁴ to 10¹⁴ pfu.

10. The method according to claim 9, wherein said recombinant virus is administered by an intratracheal route and said recombinant virus comprises from 10⁶ to 10¹⁰ pfu.

11. The method according to claim 1, wherein said vector which comprises a nucleic acid encoding at least one angiogenic factor is co-administered to said individual with at least one compatible and pharmaceutically acceptable adjuvant.

12. A pharmaceutical composition comprising a defective recombinant vector which comprises a nucleic acid encoding at least one angiogenic factor, wherein said composition is formulated for intratracheal administration.

13. The pharmaceutical composition according to claim 12, wherein said composition comprises at least one compatible and pharmaceutically acceptable adjuvant for intratracheal administration.

14. The pharmaceutical composition according to claim 12, wherein said defective recombinant vector comprises from 10⁴ to 10¹⁴ pfu.

15. The pharmaceutical composition according to claim 14, wherein said defective recombinant vector comprises from 10⁶ to 10¹⁰ pfu.
